(19) United States
(12) Patent Application Publication
(10) Pub. No.: US 2025/0318922 A1
(43) Pub. Date: Oct. 16, 2025

(54) A MEDICAL DEVICE FOR IMPROVING FUNCTION OF A HEART VALVE

(71) Applicant: HVR CARDIO OY, Espoo (FI)

(72) Inventor: Keränen OLLI, Bjärred (SE)

(21) Appl. No.: 18/863,209

(22) PCT Filed: May 9, 2023
(86) PCT No.: PCT/FI2023/050255

§ 371 Date: Nov. 5, 2024

(30) Foreign Application Priority Data
May 9, 2022 (FI) 20225400

(51) Int. Cl. A61F 2/24

(52) U.S. Cl. CPC A61F 2/2409; A61F 2/2412; A61F 2/2427; A61F 2220/0016; A61F 2220/0075

(57) ABSTRACT

A medical arrangement (100) comprises a tissue anchoring device (130) to be introduced around an annulus (20) and an artificial leaflet (101) to be introduced together with the tissue anchoring device (130) to an inner section of a valve tissue. The artificial leaflet (101) comprises a delivery state (102) and an activated state (103), wherein in said delivery state (102) the leaflet has a reduced form so that an area of the leaflet is smaller than in said activated state (103). In the activated state (103) artificial leaflet (101) has an expanded form and covers at least a portion of biological leaflets (22). The artificial leaflet (101) is transferable from the said delivery state (102) to the activated state (103) at the same, or even by a same back and forth movement, when the tissue anchoring device (130) is introduced to surround at least a circular portion of the annulus (20).

### TECHNICAL FIELD OF THE INVENTION

The invention relates to a medical device for improving function of a heart valve. Especially the invention relates to a medical device for improving the function of leaflets or introducing an artificial leaflet to the heart or replacing an original leaflet or leaflets at least partly with the artificial leaflet.

### BACKGROUND OF THE INVENTION

FIG. 1A illustrates a portion of the heart 12, the mitral valve 18, and the left ventricle 14. The mitral valve is at its boundary circumferenced by an annulus 20. The valve has two cusps or leaflets 22, 24. Each of these cusps or leaflets 22, 24 are connected to a respective papillary muscle 27, 29 via their respective connecting chordae 26, 28. In normal healthy individuals the free edges of the opposing leaflets will close the valve by coaptation. However, for some individuals the closure is not complete, which results in a regurgitation, also called valvular insufficiency, i.e. back flow of blood to the left atrium making the heart less effective and with potentially severe consequences for the patient. FIG. 1B illustrates a mitral valve 18, in which the leaflets 22, 24 do not close properly. This commonly occurs when the annulus 20 becomes dilated. One surgical procedure to correct this is to remove a portion of the leaflet 24 and stitch the cut edges together with one another. The procedure will pull back the annulus 20 to a more normal position. However, the strength of the leaflet 24 is altered. Similar problems with a less effective heart function occur if one or both leaflets are perforated to such an extent that blood is flowing towards the left atrium, although the leaflets close properly.

In some conditions of degenerated heart function, the leaflets do not present a solid surface, as in a degenerative valve disease. The leaflet could also be perforated, with one or several holes, where the blood can flow backwards into the atrium. Another possibility is that the leaflet is ruptured, most commonly at an edge of a leaflet, resulting in an incomplete coaptation.

There are artificial leaflets known from the prior art, such as disclosed in EP2591754 (applicant's own publication), where an artificial flexible leaflet reinforcement patch is advantageously connected to an anchoring unit for anchoring the artificial flexible leaflet reinforcement patch to the annulus and additionally for arranging the artificial flexible leaflet reinforcement patch in juxtaposition with the leaflet.

Even if the artificial flexible leaflet reinforcement patch has numerous advantages, there are however still some challenges relating to the known prior art artificial leaflets, such as to deliver them to the atrium or ventricle in via a catheter and place them accurately in connection with the annulus or natural leaflets. In addition, one should know an area of the surface to be replaced beforehand.

Hence, a medical arrangement with an artificial leaflet for delivering the artificial leaflet to the heart would be advantageous and, in particular, such a medical arrangement allowing for repair of one or more leaflets of a heart valve, or other related anatomical leaflet structures.

### SUMMARY OF THE INVENTION

An object of the invention is to alleviate and eliminate the problems relating to the known prior art. Especially the object of the invention is to provide a medical arrangement, by which an artificial leaflet can be introduced to the heart via a catheter advantageously in a controlled manner so that to control the introduction to place the artificial leaflet in a correct place.

The object of the invention can be achieved by the features of independent claims.

The invention relates to a medical arrangement for improving function of a heart valve according to claim 1. In addition, the invention relates to a method for improving function of a heart valve according to claim 20.

According to an embodiment of the invention a medical arrangement for improving function of a heart valve is provided. The heart valve comprises valve tissue, including an annulus at an outer section of the valve tissue and in a normal healthy state a plurality of biological leaflets at an inner section of said valve tissue, as is depicted already in FIGS. 1A and 1B. The medical arrangement comprises a tissue anchoring device to be introduced to or around the annulus at least partially so that the tissue anchoring device surrounds at least a circular portion of the annulus. The tissue anchoring device has advantageously a delivery state (essentially elongated form), so that it can be delivered though a catheter, for example, and an activated state where is takes the form to be used in use, such as a form of an arc, a ring or a helix ring, for example. The tissue anchoring device may comprise e.g. shape memory material in order to take the form in use when it is exposed to a suitable temperature, such as a blood circulation temperature. The tissue anchoring device may have a portion or portions to be placed above or below or both sides of the annulus as can be seen e.g. in FIG. 2. It is to be understood that the invention is not limited to any special forms of the tissue anchoring device.

According to an embodiment of the invention the medical arrangement comprises also an artificial leaflet to be introduced to the inner section of said valve tissue or in more precisely on the place where at least one of the biological leaflets locates in the normal healthy state. The artificial leaflet comprises also a delivery state and an activated state, and the artificial leaflet is transferable from the delivery state to the activated state when it is introduced to its place in the heart. In addition, in some embodiments the artificial leaflet can be transferred also to the delivery state from the activated state at least partially (like one segment or portion of it), which might help for example in positioning the artificial leaflet. Typically, this can be done when the artificial leaflet is not yet disconnected from the introduction devices, such as a pusher or the like.

In said delivery state the leaflet has a reduced form so that an area of the leaflet is smaller than in said activated state. In the activated state the leaflet has an expanded form so that the area of the leaflet is bigger than in said delivery state and wherein the leaflet in the expanded form covers at least a portion of the biological leaflets or even the entire biological leaflets to be fixed. Thus, also partial replace of biological leaflet is possible. According to the invention the artificial leaflet may be folded or reeled so to achieve the delivery state. For example, in the folded form distal and proximal ends of the artificial leaflet are closer to each other and in the activated state the folded form is opened so that the distal and proximal ends of the artificial leaflet are moved farther each other. In addition, for example in the reeled form the artificial leaflet is reeled into a roll (such as a roller blind) and in the in the activated state the reeled form is opened.

According to an embodiment the artificial leaflet is fixed to the tissue anchoring device so that the tissue anchoring device transports or introduces the artificial leaflet to the heart at the same when the tissue anchoring device is pushed to the heart via the catheter. The introduction of the artificial leaflet can be implemented e.g. so that the distal end of the artificial leaflet is fixed to the distal portion of the tissue anchoring device and next to that also the proximal end of the artificial leaflet is fixed in a detachable manner. Thus, when the tissue anchoring device is introduced to the heart so that the distal portion of the tissue anchoring device and also the proximal end of the artificial leaflet (fixed to the distal portion of the tissue anchoring device) meet a proximal area of the biological leaflet, so the area where the tissue anchoring device is coming from during the introduction, the proximal end of the artificial leaflet is released from the distal portion of the tissue anchoring device.

The proximal end of the artificial leaflet can be attached or fixed to the annulus for example by methods known from prior art, such as by suturing, or by a friction between the artificial leaflet and the tissue at the proximal area of the biological leaflet. Also, the leaflet may comprise spikes or other securing members, as disclosed elsewhere in this document, where the spikes can be used to fasten the leaflet, such as edge area of the leaflet to the natural tissue, like to the annulus. Then, when the tissue anchoring device is further pushed the distal end of the artificial leaflet, which is still fixed to the distal portion of the tissue anchoring device, pulls the rest of the artificial leaflet with the tissue anchoring device and thereby transfer the artificial leaflet from the delivery state to the activated state at the same when the tissue anchoring device is introduced to surround at least a circular portion of the annulus. It is to be noted that artificial leaflet can still be transferred also to or at least towards the delivery state from the activated state by moving the tissue anchoring device backwards, because the distal end of the artificial leaflet is still fixed to the distal portion of the tissue anchoring device and because the proximal end of the artificial leaflet is disconnected from the tissue anchoring device and fixed to the annulus or other tissue.

It is to be noted that the artificial leaflet may have numerous of slidable fixing means, such as a loop or the like, via which the artificial leaflet is coupled with the tissue anchoring device and so that only the distal end of the artificial leaflet is fixed in the proximal portion of the tissue anchoring device. The slidable fixing means are configured to slide along the tissue anchoring device when the tissue anchoring device is pushed further and at the same the proximal end of the artificial leaflet is fixed to the proximal area of the biological leaflet, causing the artificial leaflet to transform to the activated state from the delivery state. The slidable fixing means can be separate rings or achieved by extending the material of the artificial leaflet around the tissue anchoring device in order to form a long loop, for example, so to form slidable fixing means from the extended material of the artificial leaflet. Then no separate slidable fixing means are needed which might decrease for example costs and make the structure of the entire arrangement simpler. In addition, in some cases stuck of the slidable fixing means to the tissue can be avoided by the slidable fixing means of the extended material of the artificial leaflet, because the slidable fixing means is much smoother with no separate rings and thus lumps or separate protrusions.

Anyway, according to embodiments of the invention the slidable fixing means can be implemented e.g. by the separate rings or by extending the material of the artificial leaflet around the tissue anchoring device to form a long loop so that the loop is configured to slide along the tissue anchoring device in its longitudinal direction, as discussed above, but also other arrangements can be utilised. According to an embodiment, the tissue anchoring device may comprise a groove and the artificial leaflet protrusion members, where each of the protrusion member comprises an enlarged end portion. The enlarged end portions of the protrusion members are advantageously suitable to be provided into the groove of the tissue anchoring device so that the form of the groove prevent the slip of the enlarged end portion away from the groove but allows sliding of the enlarged end portion along the groove in the longitudinal direction of the groove. The enlarged end portion may comprise a hanger which is configured to be slide in the groove and at the same fix the artificial leaflet to the tissue anchoring device. Alternatively, the edge portion of the artificial leaflet can be folded so to have a thick portion and thus having enlarged end portion which ca be introduced to the groove and to slide in the groove and at the same fix the artificial leaflet to the tissue anchoring device.

According to an embodiment the introduction of the artificial leaflet to its position can be implemented so that the distal and proximal ends of the artificial leaflet is fixed to the distal portion of the tissue anchoring device and so that the proximal end of the artificial leaflet is fixed in a detachable manner, as discussed previously. However, now the distal portion of the tissue anchoring device is introduced to the proximal area of the biological leaflet so that both the distal and proximal ends of the artificial leaflet are still fixed to the distal portion of the tissue anchoring device. Then, when the distal end of the artificial leaflet is at the distal area of the biological leaflet, the proximal end of the artificial leaflet is released from the tissue anchoring device and the proximal end of the artificial leaflet is retracted back towards the proximal area of the biological leaflet.

The retraction of the proximal end of the artificial leaflet can be implemented e.g. by coupling a retraction means, such as a yarn or the like, in a detachable manner to the proximal end of the artificial leaflet and operate, such as pull, the retraction means e.g. outside the heart. The artificial leaflet is transferred from said delivery state to said activated state when the proximal end of the artificial leaflet is retracted essentially to the proximal area of the biological leaflet. In addition, when the proximal end of the artificial leaflet is retracted essentially to the proximal area of the biological leaflet, the retraction means is advantageously detached.

Again, at least the proximal end of the artificial leaflet can be attached or fixed to the annulus for example by methods known from prior art, such as by suturing, or by a friction between the artificial leaflet and the tissue at the proximal area of the biological leaflet.

According to an embodiment the artificial leaflet comprises a stretch material so that in the delivery state the artificial leaflet is in a rest position and in the activated state the artificial leaflet is stretched to the expanded form. The retraction can be implemented as discussed elsewhere in this document in connection with the other embodiments. According to an embodiment the artificial leaflet may also be in the delivery state in a tensioned position having potential energy (spring force) and in said activated state the potential energy of the tensioned artificial leaflet is released to cause the artificial leaflet to transform to the expanded form. Again, the artificial leaflet comprises advantageously the slidable fixing means, such as a loop or the like as discussed elsewhere in this document, via which the artificial leaflet is coupled with the tissue anchoring device. The releasing of the tensioned artificial leaflet can be implemented e.g. by a soluble fixing means which will be dissolved when exposed to the blood circulation of the heart. Alternatively, the tensioned artificial leaflet might have a pin, which is released for example by a releasing member coupled with the pin, such as a yarn. The outer edge area of the artificial leaflet can also be folded around the tissue anchoring device to achieve the slidable fixing means, as is discussed in connection with other embodiments.

According to an embodiment the artificial leaflet may comprise a shape memory material having a first shape, such as the delivery state in a first temperature, and the second shape, such as the activated state in a second temperature, whereupon the artificial leaflet is configured to take said activated state when it is exposed to the second temperature and one of the distal or proximal end is released from the tissue anchoring device.

According to an embodiment the artificial leaflet can be rolled next to the tissue anchoring device in the delivery state, whereupon the artificial leaflet can be rolled open from the tissue anchoring device when the tissue anchoring device is introduced to an appropriate location around the annulus. In this embodiment the artificial leaflet may comprise shape memory material so that in the first temperature it is in rolled form and in the second temperature in the opened form, or in the rolled form having potential energy which can be released so to transfer the artificial leaflet to the activates state. The releasing of the tensioned artificial leaflet can be implemented e.g. correspondingly as discussed elsewhere in this document in connection with the releasing of the tensioned artificial leaflet.

Alternatively, a pusher can be used, whereupon the artificial leaflet can be rolled around a pusher and opened by turning the pusher around a longitudinal axis of the pusher. In this way the rolling can be done very controllable manner. When the artificial leaflet is opened into the activated state (and advantageously also fixed to the tissue), the pusher can be retracted away.

It is to be noted that the artificial leaflet can be attached to the tissue e.g. by friction or by suturing the surrounding portion of the artificial leaflet to the annulus or even to around the tissue anchoring device. Further, it is to be noted that in some embodiments the tissue anchoring device may comprise a securing member, such as a sharp tooth, so to secure the artificial leaflet to the activated state when the artificial leaflet is expanded to the activated state. The securing member, like a tooth, may locate e.g. at the distal portion and/or the proximal portion of the tissue anchoring device, depending e.g. whether the end of the artificial leaflet to be attached is a distal or proximal end. In addition, the angle of the tooth is arranged so that the artificial leaflet can be expanded into a first direction along the tissue anchoring device but so that the tooth secures the artificial leaflet and prevents its movement to a second, opposite direction.

According to an embodiment, the artificial leaflet may comprise a number of securing members, such as a sharp tooth or spikes, so to secure the artificial leaflet to the tissue when the artificial leaflet is expanded to the activated state. The securing members may be implemented in different ways, like by memory material so that the securing members are inside the artificial leaflet at least partly and when the artificial leaflet is in its position and expanded to the activated state, the memory of the securing members will expand the securing member and thereby introduce and secure the securing members and thus the artificial leaflet to the tissue. Also, an angle of the securing members may be arranged so that during the introduction of the artificial leaflet, so when the artificial leaflet is in its delivery state, the angle of the securing members is small (advantageously less than 30° or even less than) 10° so that the free ends of the securing members point towards the proximal end of the tissue anchoring device. When the artificial leaflet is expanded to the activated state, the angle of the securing members is increased (advantageously more than 45° or even more than 60°) so that the free ends of the securing members point towards the tissue thereby biting down to the tissue and securing the artificial leaflet to the tissue. The change of the angle of the securing members may be achieved by the shape memory material, for example, but also other methods can be used, such as mechanically change the angles, like pulling a yarn connected to the securing members. It is to be noted that the whole edge area of the artificial leaflet may comprise the securing members, but advantageously at least the posterior leaflet comprises the securing members. It has been noted that the securing members only in the posterior leaflet can secure the whole artificial leaflet well enough. In addition, the securing members of the artificial leaflet can secure the artificial leaflet to the tissue, but at the same it also strengthen the securing effect of the tissue anchoring device.

In many of the above embodiments the tissue anchoring device is used for delivering the artificial leaflet to the heart so that the distal and/or proximal end is fixed to the tissue anchoring device, but alternatively a pusher can be used for delivering or at least help the delivering the artificial leaflet to the heart or transferring the artificial leaflet from the delivery state to the activated state. The pusher can be a separate pusher dedicated to deliver only the artificial leaflet or a pusher to be used for delivering also the tissue anchoring device advantageously at the same.

For example, the pusher can be used to introduce the tissue anchoring device together with the artificial leaflet, whereupon the pusher can be coupled either with the proximal or distal end of the tissue anchoring device. If the pusher is coupled with the distal end of the tissue anchoring device, the distal end portion of the artificial leaflet is also advantageously coupled with the distal portion of the tissue anchoring device or the distal portion of the pusher. In addition, also the proximal end of the artificial leaflet is coupled with the proximal portion of the tissue anchoring device or the distal portion of the pusher next to the distal end of the artificial leaflet during introduction. Then, the proximal end of the artificial leaflet can be released during introduction the tissue anchoring device, when the proximal end of the artificial leaflet is at the proximal area of the biological leaflet so the area where the tissue anchoring device is coming from during the introduction, and thus when the tissue anchoring device is delivered further, the distal portion of the tissue anchoring device pulls the distal end of the artificial leaflet and transfer it into the activate state, similarly as discussed elsewhere in this document. It is also possible that the proximal end of the artificial leaflet is released only when the distal portion of the artificial leaflet and the distal portion of the tissue anchoring device is delivered to their locations, and in this case the proximal end of the artificial leaflet will be retracted backwards by the pusher and released from the pusher when it is essentially at the proximal area of the biological leaflet. Advantageously, the pusher can transfer the artificial leaflet from the delivery state to the activated state by the same back and forth movement when the tissue anchoring device is introduced to surround at least a circular portion of the annulus. The disconnection of the artificial leaflet from the pusher can be implemented e.g. with a pin, which can be released e.g. moving the pin by a rotational movement of the pusher. In addition, the pusher can be connected to the tissue anchoring device so that when the pusher is rotated an angle around its longitudinal axis, the tissue anchoring device and the pusher is disconnected.

If the pusher is coupled with the proximal end of the tissue anchoring device, the distal and proximal ends of the artificial leaflet are advantageously coupled to the distal end of the tissue anchoring device. Thus, when the proximal end of the artificial leaflet is at the proximal area of the biological leaflet, the proximal end of the artificial leaflet can be released from the tissue anchoring device so that the proximal end of the artificial leaflet is left, or can be even fixed, to the tissue, such as to the annulus, and the artificial leaflet is transferred to the activated state when the tissue anchoring device is pushed further towards the distal portion of the biological leaflet. Alternative, the distal and proximal ends of the artificial leaflet can be pushed with the tissue anchoring device to the distal area of the biological leaflet and the proximal end of the artificial leaflet is released at the distal area of the biological leaflet. Then, the proximal end of the artificial leaflet is retracted e.g. by the retraction means, such as disclosed elsewhere in this document, or the proximal end of the artificial leaflet can be coupled to the distal end of the pusher so that when the pusher is released from the tissue anchoring device and retracted away, the pusher will retract also the proximal end of the artificial leaflet backwards to the proximal area of the biological leaflet. Then, when the proximal end of the artificial leaflet is at the proximal area of the biological leaflet, the pusher or retraction means is detached from the artificial leaflet and the proximal end of the artificial leaflet can be fixed to the tissue as disclosed elsewhere in this document.

According to an embodiment also the catheter can be used as the retraction means. For example, the distal end of the artificial leaflet can be delivered to the distal portion of the biological leaflet either by the tissue anchoring device, pusher or the catheter, and the proximal end of the artificial leaflet is retracted by the catheter when the catheter is retracted away from the heart. The proximal end of the artificial leaflet is advantageously coupled to the distal end of the catheter in a detachable manner, such as e.g. by a yarn which can be cut, for example by a separate instrument when needed so when the proximal end of the artificial leaflet is at the proximal area of the biological leaflet.

According to an example at least two pushers can be used, one for the tissue anchoring device and another to the artificial leaflet. According to an embodiment, the distal end of the artificial leaflet can be coupled to the distal end of the pusher, and when the distal end of the artificial leaflet is delivered to the distal area of the biological leaflet the distal end of the artificial leaflet is released from the pusher and the pusher is used to retract the proximal end of the artificial leaflet backwards to the proximal area of the biological leaflet. Alternatively, the proximal end of the artificial leaflet can be released or left to the proximal area of the biological leaflet from the pusher when the pusher is still pushed forward and introduce the distal end of the of the artificial leaflet towards the distal area of the biological leaflet, and thereby transfer the artificial leaflet from the delivery state to the activated state.

When the tissue anchoring device is introduced to its location, it is advantageously fixed to the annulus by the fixing means, such as e.g. suturing. The artificial leaflet can be fixed at the same or separately. According to an embodiment, the tissue anchoring device may be used to press the artificial leaflet between the annulus and the tissue anchoring device by a sprig force of the tissue anchoring device, especially if the tissue anchoring device comprises shape memory material, and thereby secure and prevent any unwanted movement of the artificial leaflet between the delivery and activated states after the artificial leaflet is expanded to the activated state.

The tissue anchoring unit, when in use, may also be used to secure the artificial leaflet to the adjacent tissue and/or annulus, in such a manner that the artificial leaflet is prevented from dislocating itself, without loss of the desired function of the artificial leaflet. Hence, the artificial leaflet will be less affected by the forces of the pulsatile blood flow and the dynamics of the beating heart, which would dislocate the artificial leaflet otherwise.

Even if only one artificial leaflet is described above, it is also clear that two artificial leaflets can be delivered in manner described here for one artificial leaflet. If two artificial leaflets are introduced, they are advantageously introduced sequentially and if the tissue anchoring device is used, arranged sequentially to the tissue anchoring device. If two artificial leaflets are introduced, the first leaflet is advantageously the anterior leaflet, which is also a dominant leaflet for example in a mitral valve, and the second one is a posterior leaflet. The free section or end of the leaflet is configured to be oriented towards the inner section of the valve tissue when in use. In addition, when the two artificial leaflets are used or at least one of the biological leaflets is still left, the free end of the artificial leaflet is configured to overlap another leaflet's free end to form a coaptation surface area.

The artificial leaflet may comprise e.g. leather, tissue, such as pericardium tissue or artificial tissue, fabric, such as polyester or coretex, or porosity material or fabric supporting endothelial surface growth or mesh like surface thereby enabling the artificial leaflet to grow into a natural biological leaflet in use where the artificial leaflet is in juxtaposition against the biological leaflet.

The embodiment described offer many advantages over the known prior art, such as easy way to introduce an artificial leaflet to the area of the biological leaflet via a catheter, because in the delivery state the artificial leaflet takes much smaller area or volume when in the activated state. In addition, the invention allows to repair or replace the entire biological leaflet or only portion of it, such as only damaged area of the biological leaflet, locate the artificial leaflet accurately and in a controlled manner like leaving the distal or proximal end of the artificial leaflet accurately to a desired position and opening (transferring to the activated state) the artificial leaflet to the desired direction.

In addition, the introduction of the artificial leaflet can be done even if the heart is beating and no cardiopulmonary bypass or machines (heart-lung machine) to take over the function of the heart and lungs during surgery is needed. Thus, also the healing process after the surgery is faster with less risks to any symptoms, for example.

The exemplary embodiments presented in this text are not to be interpreted to pose limitations to the applicability of the appended claims. The verb “to comprise” is used in this text as an open limitation that does not exclude the existence of also unrecited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

### BRIEF DESCRIPTION OF THE DRAWINGS

Next the invention will be described in greater detail with reference to exemplary embodiments in accordance with the accompanying drawings, in which:

FIGS. 1A-1B illustrate schematically a portion of a heart and mitral valve,

FIG. 2 illustrates an exemplary tissue anchoring device for securing the artificial leaflet to the adjacent tissue and/or annulus according to an advantageous embodiment of the invention,

FIG. 3 illustrates an example of the tissue anchoring device and the artificial leaflet in the delivery state coupled to the tissue anchoring device according to an advantageous embodiment of the invention,

FIG. 4 illustrates an example of the medical arrangement, where the artificial leaflet is in the activated state according to an advantageous embodiment of the invention,

FIGS. 5-7 illustrates an example of the medical arrangement, where the artificial leaflet is delivered by the tissue anchoring device according to an advantageous embodiment of the invention,

FIGS. 8-10 illustrates an example of the medical arrangement, where the artificial leaflet is delivered by the pusher according to an advantageous embodiment of the invention,

FIGS. 11-13 illustrates an example of the medical arrangement, where the artificial leaflet is transported from the delivery state to the activated state by a retracting device according to an advantageous embodiment of the invention,

FIG. 14-16 illustrates an example of the medical arrangement, where the artificial leaflet is delivered by the tissue anchoring device and transferred to the activated state by a catheter according to an advantageous embodiment of the invention,

FIGS. 17-19 illustrates an example of fixing of the artificial leaflet by the tissue anchoring device according to an advantageous embodiment of the invention,

FIGS. 20-22 illustrates an example of the medical arrangement, where the artificial leaflet is stretchable material and how it is stretched to the activated state according to an advantageous embodiment of the invention,

FIGS. 23-25 illustrates an example of the medical arrangement, where the artificial leaflet is rolled in the delivery state and the roll is opened to transfer the artificial leaflet to the activated state according to an advantageous embodiment of the invention,

FIG. 26 illustrates an example of the tissue anchoring device in an elongated form in the delivery state and with two artificial leaflets according to an advantageous embodiment of the invention,

FIGS. 27-29 illustrates examples of the slidable fixing means according to an advantageous embodiment of the invention, and

FIGS. 30-31 illustrates examples of the securing members for securing the artificial leaflet according to an advantageous embodiment of the invention.

### DETAILED DESCRIPTION

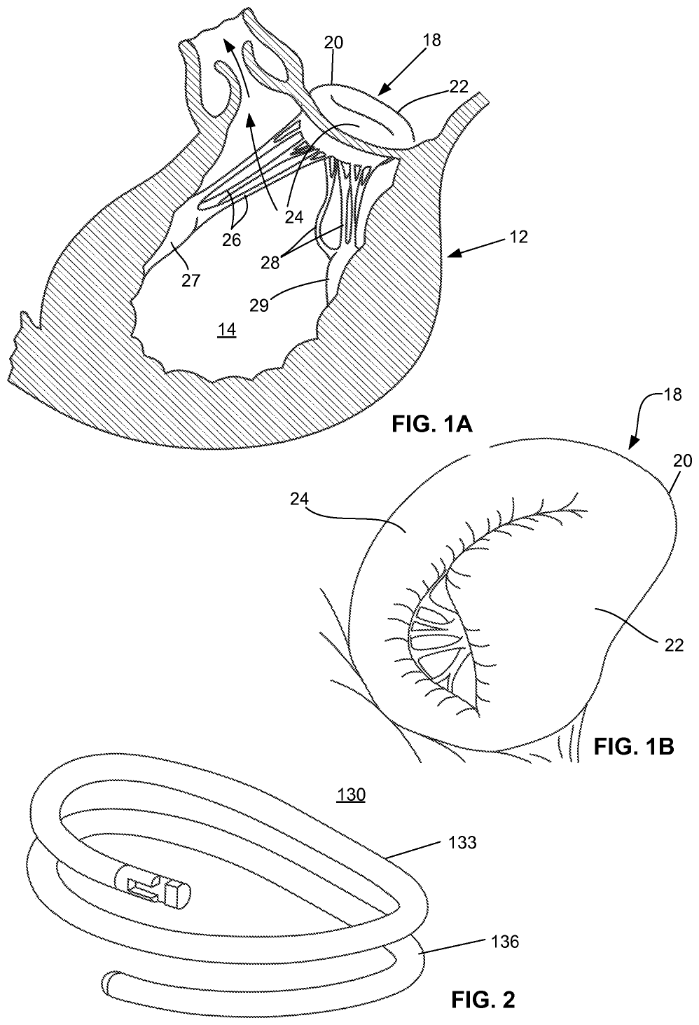

FIGS. 1A-1B illustrating schematically a portion of a heart and mitral valve are already discloses in the connection with the background portion of the invention.

FIG. 2 illustrates an exemplary tissue anchoring device 130 for securing the artificial leaflets 101 (not shown in FIG. 2) to the adjacent tissue and/or annulus 20. The tissue anchoring device is configured to abut a first side of the heart valve and configured to abut a first side of the heart valve. The exemplary tissue anchoring device 130 comprises a first loop-shaped support structure 133. The first loop-shaped support structure 133 comprises first and second sections and the first and second sections are arranged to locate (in use so in an activated state) essentially at the opposite sides of the loop-shaped support structure 133 when the tissue anchoring unit 130 takes the loop formation in an activated state.

The first loop-shaped support structure 133 is advantageously anatomical D-shape or anatomical kidney shape having essentially an elongated portion and a curved portion. The elongated portion is configured to be faced to the anterior side of the mitral valve 18 and the curved portion to the posterior side of the mitral valve 18, when in use. The first section of the tissue anchoring unit 130 is located essentially at the elongated portion to which the artificial leaflet 101 is fastened or along which the slidable fixing means 106 is configured to slide when the artificial leaflet is transferred from the delivery state to the activated state.

The tissue anchoring device 130 may comprise also a second loop-shaped support structure 136 forming with the first loop-shaped support structure a continuous helix structure, when being in activated state (as is depicted in FIG. 2). The second loop-shaped support structure is configured to abut a second, opposite, side of the valve to thereby trap a portion of the valve tissue 20 between the second and the first support structures 133, 136. However, it is to be understood that the tissue anchoring device 130 used in the invention may have only one of the support structures 133, 136 (so only one loop) or only a circular structure, such as an arc.

The tissue anchoring device advantageously comprises a shape memory material having a first shape, such as the elongated form of the delivery state in a first temperature (as can be seen e.g. in FIG. 26), and the second shape, such as the loop-shaped form in a second temperature (as is the case in FIG. 2). The second temperature corresponds advantageously essentially the body temperature, whereupon the tissue anchoring unit takes the second shape, so the loop-shaped form, when introduced for example with the blood flow in the atrium.

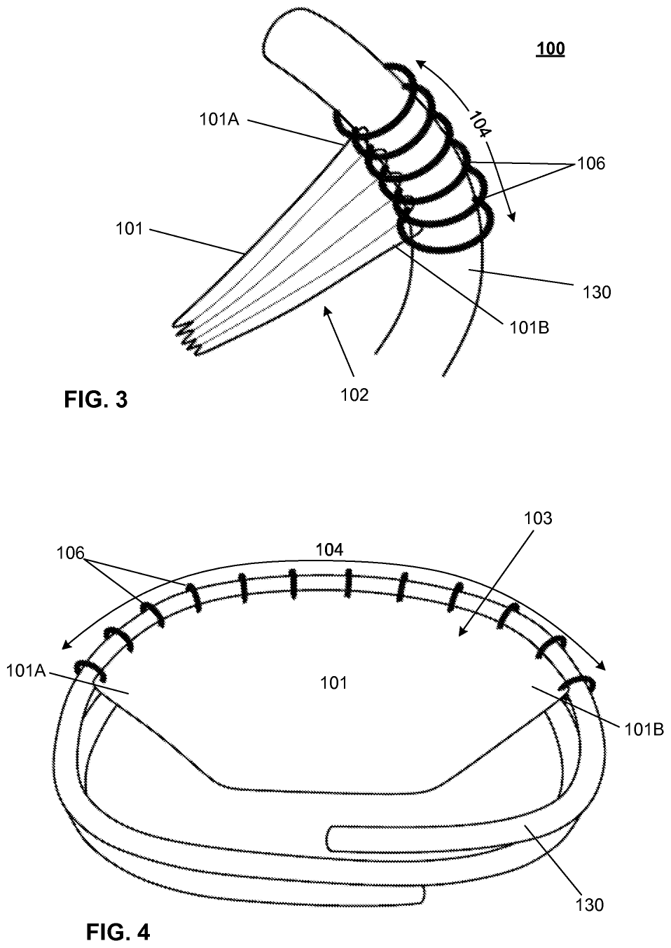

FIGS. 3 and 4 illustrate an example of the tissue anchoring device 130 and the artificial leaflet 101. In FIG. 3 the artificial leaflet 101 is in the delivery state 102 and coupled to the tissue anchoring device 130. In the delivery state 102 the distal end 101A and the proximal end 101B of the artificial leaflet 101 is fixed to the distal portion 130A of the tissue anchoring device 130 and the distance 104 of the distal end 101A and the proximal end 101B of the artificial leaflet 101 is smaller than in the activated state (see FIG. 4). FIG. 4 illustrates the situation where the artificial leaflet 101 is transferred to the activated state 103 according to an advantageous embodiment of the invention, and there the distance 104 of the distal end 101A and the proximal end 101B of the artificial leaflet 101 is bigger than in the deliverer state (see FIG. 3). The transfer of the artificial leaflet from the delivery state 102 to the active state 103 can be done in numerous ways, as is described elsewhere in this document.

In the delivery state 102 the leaflet has a reduced form so that an area of the leaflet is smaller than in the activated state 103. In the activated state 103 the leaflet 101 has an expanded form so that the area of the leaflet 101 is bigger than in the delivery state 102 and wherein the leaflet 101 in the expanded form covers at least a portion of the biological leaflets or even the entire biological leaflets 22, 24 to be fixed, as can be seen in FIGS. 3 and 4.

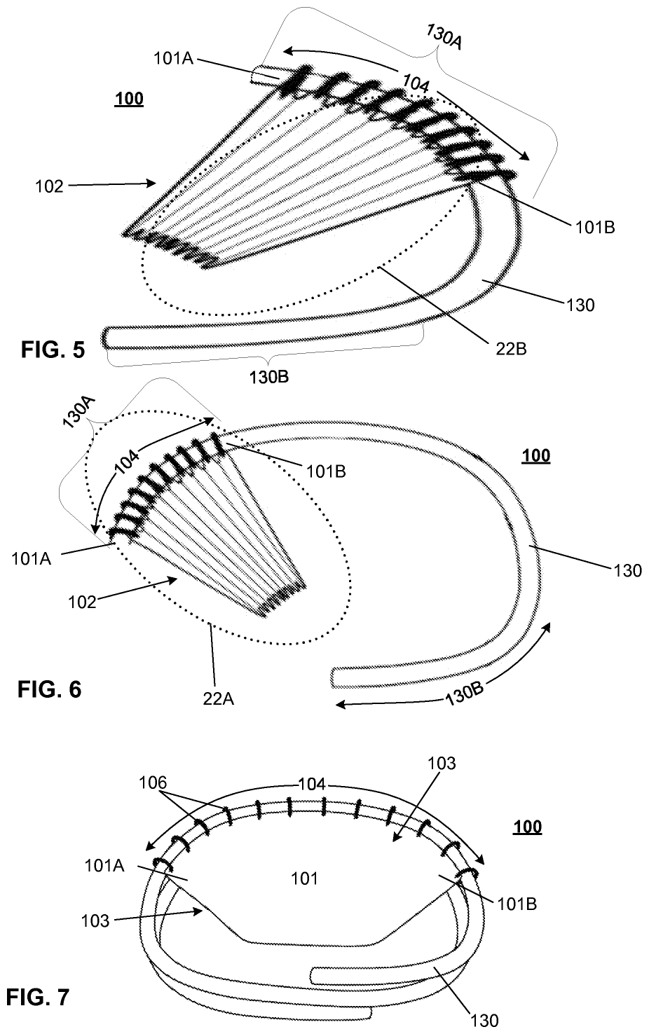

FIGS. 5-7 illustrates an example of the medical arrangement 100, where the artificial leaflet 101 is delivered by the tissue anchoring device 130 according to an advantageous embodiment of the invention. The artificial leaflet 101 is fixed to the tissue anchoring device 130 so that the tissue anchoring device 130 transports the artificial leaflet 101 to the heart at the same when the tissue anchoring device 130 is introduced, advantageously pushed to the heart via the catheter (not shown). In FIG. 5 the distal end 101A of the artificial leaflet 101 is fixed to the distal portion 130A of the tissue anchoring device and next to that also the proximal end 101B of the artificial leaflet 101 is fixed in a detachable manner. Thus, when the tissue anchoring device 130 is introduced to the heart so that the distal portion 130A of the tissue anchoring device 130 and also the proximal end 101B of the artificial leaflet 101 meet a proximal area 22B of the biological leaflet, the proximal end 101B of the artificial leaflet is released from the distal portion 130A of the tissue anchoring device 130. The proximal end 101B of the artificial leaflet 101 is attached to the annulus and/or to the proximal area 22B of the biological leaflet and the tissue anchoring device 130 is further pushed so that the distal end 101A of the artificial leaflet 101 is still fixed to the distal portion 130A of the tissue anchoring device 130. When the tissue anchoring device 130 is delivered further, the distal end 101A of the artificial leaflet 101 drags the rest of the artificial leaflet 101 and thereby transfer the artificial leaflet 101 from the delivery state 102 to the activated state 103, as is the case in FIG. 7.

Alternatively, the distal and proximal ends 101A, 101B of the artificial leaflet 101 is fixed to the distal portion 130A of the tissue anchoring device 130 and delivered over the proximal area 22B of the biological leaflet and again further to the distal area 22A of the biological leaflet by the tissue anchoring device 130. The proximal end 101B of the artificial leaflet 101 is fixed in a detachable manner, as discussed previously, and after delivering the distal portion 130A of the tissue anchoring device 130 and the distal portion 101A of the artificial leaflet 101 to the distal area 22A of the biological leaflet, the proximal end 101B of the artificial leaflet 101 is released from the tissue anchoring device 130 and the proximal end 101B of the artificial leaflet 101 is retracted backwards the proximal area 22B of the biological leaflet, as is the case e.g.

in FIG. 6.

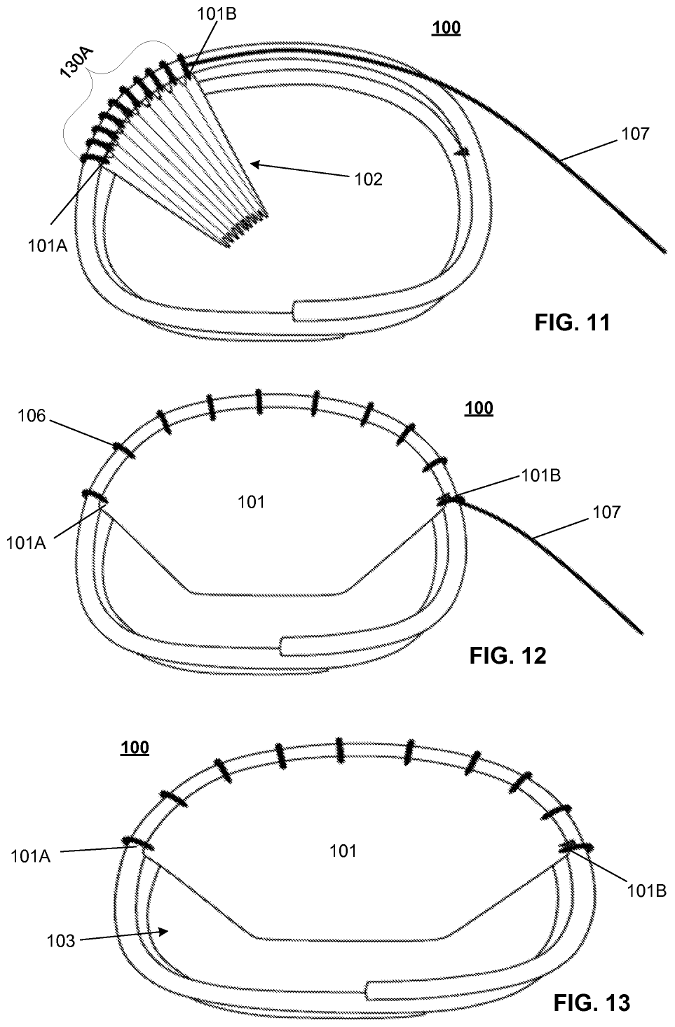

The retraction of the proximal end 101B of the artificial leaflet 101 can be implemented e.g. by coupling a retraction device 107, such as a yarn or the like, as is illustrated in FIGS. 11-13, where the proximal end 101B of the artificial leaflet 101 is retracted back to the proximal portion 22B of the biological leaflet by the retraction means 107 so to transfer the artificial leaflet 101 from the delivery state 102 (FIG. 11) to the activate state 103 (FIG. 12).

The retraction device 107 is advantageously detached when the proximal end 101B of the artificial leaflet 101 is retracted essentially to the proximal area 22B of the biological leaflet. The distal end 101A of the artificial leaflet 101 is advantageously fixed to the distal end 130A tissue anchoring device 130 or to the distal area 22A of the biological leaflet, such as to the annulus, and in the activate state 103 also the proximal end 101B of the artificial leaflet 101 is attached or fixed to the tissue anchoring device 130 and/or to the proximal area 22B of the biological leaflet, such as to the annulus for example by methods known from prior art, such as by suturing, or by a friction between the artificial leaflet and the tissue at the proximal area of the biological leaflet.

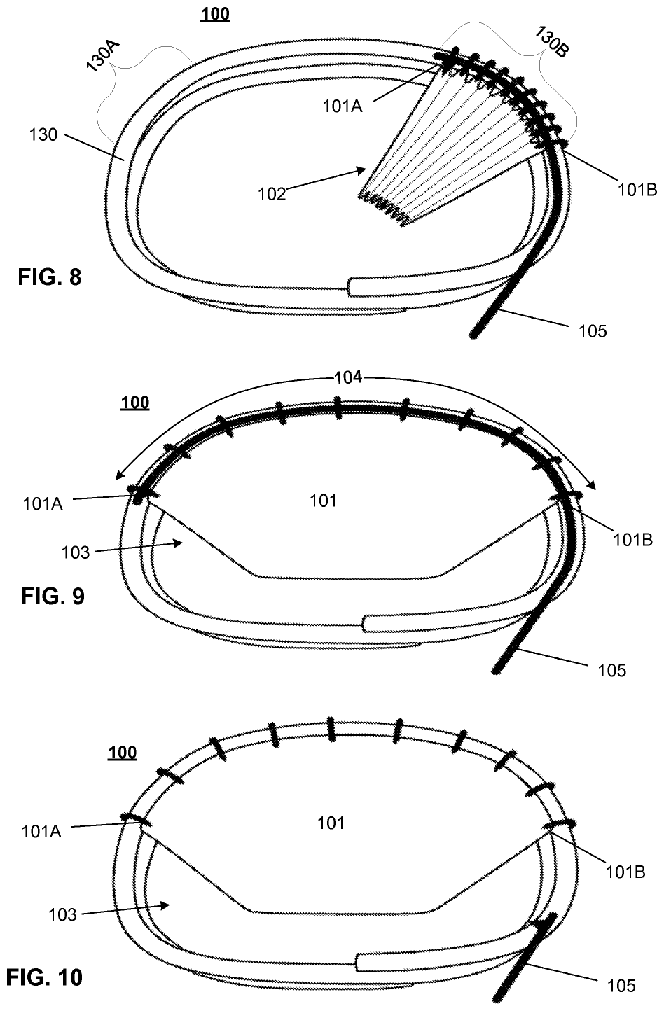

FIGS. 8-10 illustrates an example of the medical arrangement 100, where the artificial leaflet 101 is delivered by the pusher 105 according to an advantageous embodiment of the invention. The pusher 105 can be a separate pusher dedicated to deliver only the artificial leaflet 101 or a pusher to be used for delivering also the tissue anchoring device advantageously at the same when delivering the artificial leaflet.

In these examples illustrated in FIGS. 8-11, the proximal end 101B of the artificial leaflet 101 is released from the pusher 105 when the proximal end 101B of the artificial leaflet 101 is at the proximal area 22B of the biological leaflet. Thus, when the distal end 101A of the artificial leaflet 101 is pushed further by the pusher 106 to the distal area 22A of the biological leaflet, the distal end 101A of the artificial leaflet 101 will drag the rest of the artificial leaflet 101 and thereby transfers the artificial leaflet 101 from the delivery state 102 to the active state 103, as can be seen on FIG. 9. When the artificial leaflet 101 is opened or transferred to the active state 103, the pusher 105 is detached from the distal end 101A of the artificial leaflet 101 and retracted away (FIG. 10). Similarly, the pusher 105 can be used to deliver the entire artificial leaflet 101 to the distal portion area 22B of the biological leaflet, release the distal end 101A of the artificial leaflet 101 and retract the proximal end 101B of the artificial leaflet 101 towards and to the proximal area 22B of biological leaflet when the pusher 105 is retracted away and release also the proximal end 101B of the artificial leaflet 101 when the proximal end 101B of the artificial leaflet 101 is at the proximal area 22B of the biological leaflet, thus transferring the artificial leaflet 101 from the delivery state 102 to the active state 103. It is to be noted that a catheter is advantageously used for covering and facilitating the delivery operation of the artificial leaflet 101, as well as also the delivery operation of the pusher and tissue anchoring device 130, even if not shown in FIGS. 3-13-

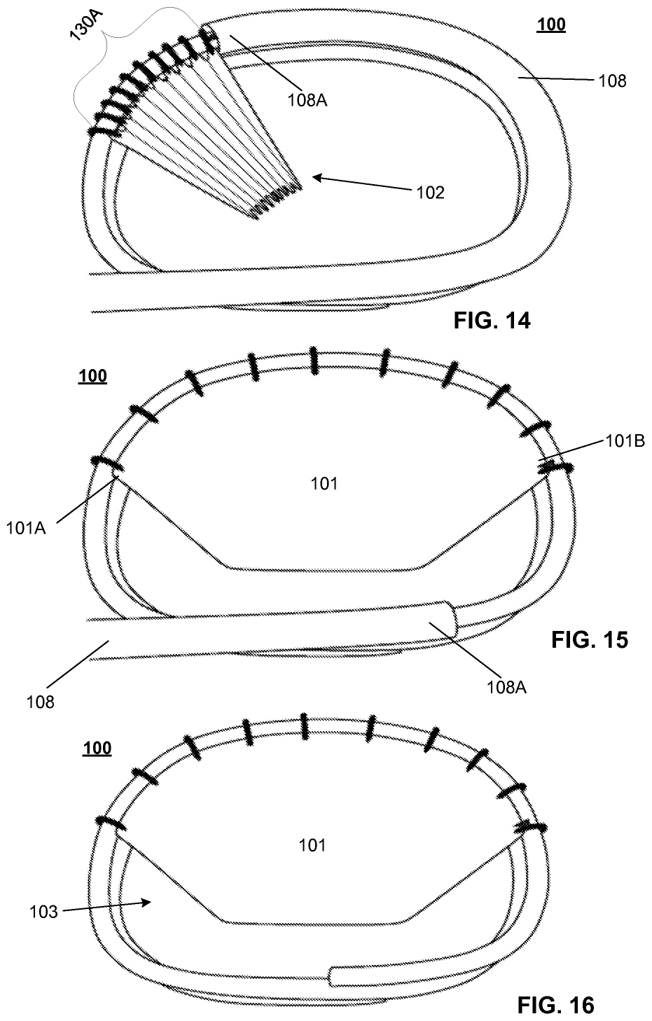

The catheter 108 can also be used for facilitating the delivery or transfer of the artificial leaflet 101 to the active state, such as is depicted in Figures FIG. 14-16, where the distal end 101A of the artificial leaflet 101 is delivered to the distal portion 22A of the biological leaflet either by the tissue anchoring device 130, pusher 105 or the catheter 108, and the proximal end 101B of the artificial leaflet 101 is retracted by the catheter 108 when the catheter 108 is retracted away from the heart. The proximal end 101B of the artificial leaflet 101 is advantageously coupled to the distal end 108A of the catheter in a detachable manner, such as e.g. by a yarn which can be cut, for example by a separate instrument (not shown) when needed so when the proximal end 101B of the artificial leaflet 101 is at the proximal area 22B of the biological leaflet.

The artificial leaflet 101 may have numerous of slidable fixing means 106, such as a loop or the like, via which the artificial leaflet 101 is coupled with the tissue anchoring device 130 in a slidable manner so that when the artificial leaflet 101 is transferred from the delivery state 102 to the active state 103 and at least one end of the artificial leaflet 101 is moved, it will slide along the tissue anchoring device 130 via the slidable fixing means 106. In addition, when the artificial leaflet 101 is transferred to the activate state 103, the slidable fixing means 106 hold the artificial leaflet 101 in the tissue anchoring device so that the artificial leaflet 101 does not loosen and move into unwanted position in the heart. However, as discloses elsewhere in this document, the slidable fixing means 106 may be achieved e.g. by folding the outer perimeter of the artificial leaflet over or around the tissue anchoring device so to form a loop so that that artificial leaflet can be slid along the tissue anchoring device in its axial direction.

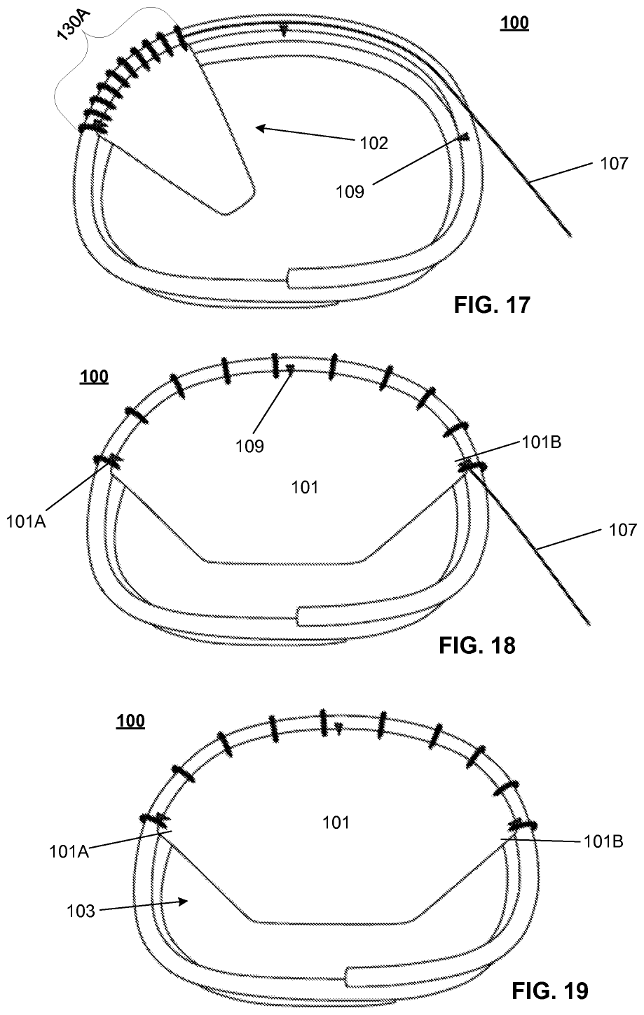

FIGS. 17-19 illustrates an example of fixing of the artificial leaflet 101 by the tissue anchoring device 130 according to an advantageous embodiment of the invention. It is to be noted that the artificial leaflet 101 can be attached to the tissue e.g. by a friction between the artificial leaflet 101 and the tissue anchoring device 130 or a friction between the artificial leaflet 101 and the tissue, as well as also by suturing the surrounding portion of the artificial leaflet to the annulus or even to around the tissue anchoring device 130. However, the tissue anchoring device 130 may also comprise a securing member 109, such as a sharp tooth, so to secure the artificial leaflet 101 to the activated state 103.

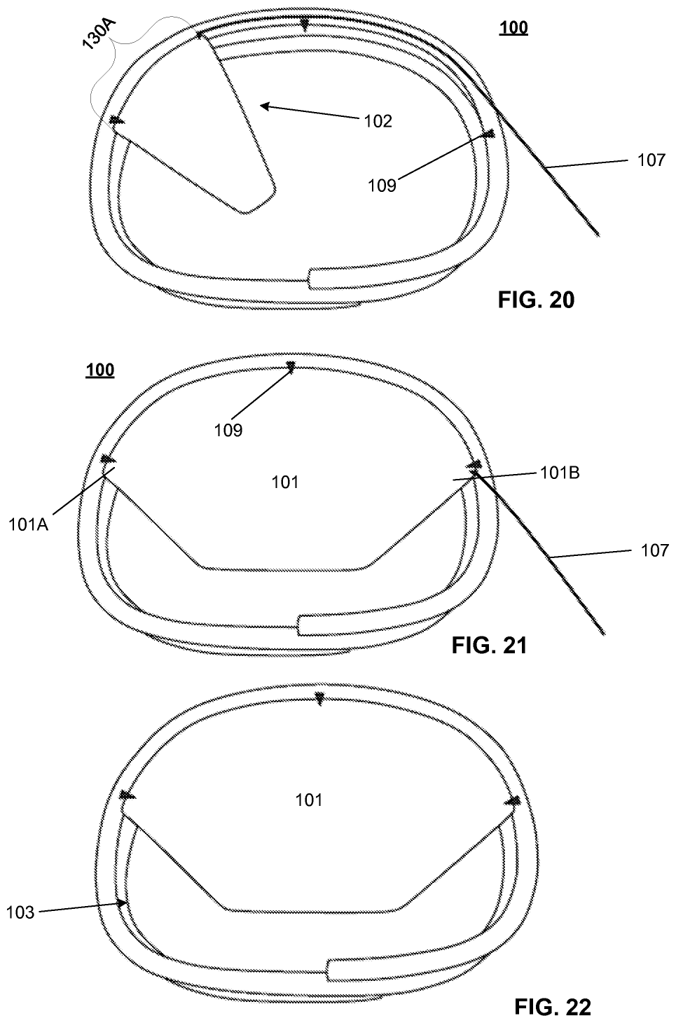

FIGS. 20-22 illustrates an example of the medical arrangement, where the artificial leaflet 101 is stretchable material and how it is stretched to the activated state 103 according to an advantageous embodiment of the invention by pulling with the retraction device 107. It is to be noted that the artificial leaflet 101 can be stretched also by the pusher 105 when the pusher is retracted away or by the catheter 108 when the catheter is retracted away. In addition, in FIGS. 20-22 the distal end 101A of the artificial leaflet 101 is first delivered and fixed to the distal area 22A of the biological leaflet and after this the proximal end 101B of the artificial leaflet 101 is stretched backwards to the proximal area 22B of the biological leaflet, but the artificial leaflet 101 can also be transferred to the active state 103 other way around so that the proximal end 101B of the artificial leaflet 101 is fixed first to the proximal area 22B of the biological leaflet and after this the distal end 101A of the artificial leaflet 101 is stretched to the distal are of the biological leaflet.

According to an embodiment the artificial leaflet may also be in a tensioned position in the delivery state 102 so that after releasing the distal and/or proximal ends 101A, 101B will slide via the slidable fixing means 106 along the tissue anchoring device 130 so to transfer the artificial leaflet 101 to the active state 103. In addition, the same can be achieved by the artificial leaflet 101 having shape memory material so that artificial leaflet 101 have a first shape, such as the delivery state 102 in a first temperature, and the second shape, such as the activated state 103 in a second temperature, whereupon the artificial leaflet 101 takes the activated state 103 when it is exposed to the second temperature and one of the distal or proximal end 101A, 101B is released from the tissue anchoring device 130 or the pusher 105, for example.

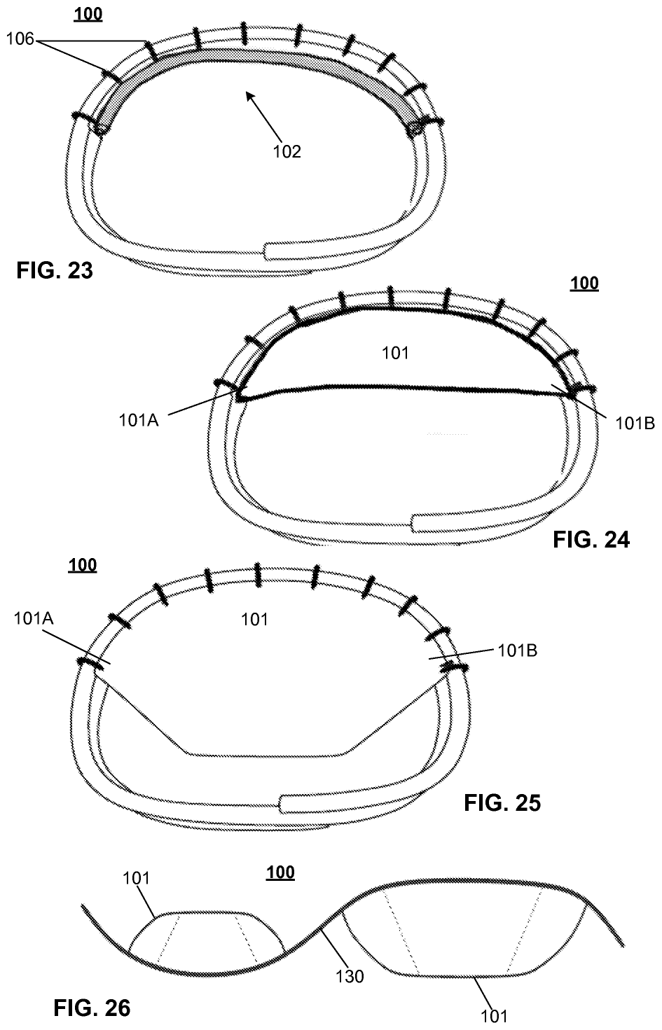

FIGS. 23-25 illustrates an example of the medical arrangement 100, where the artificial leaflet 101 is rolled in the delivery state 102 and the roll is opened to transfer the artificial leaflet 101 to the activated state 103 according to an advantageous embodiment of the invention. The artificial leaflet 101 may be e.g. in a tensioned position and when the tensioned is released the roll is opened and the artificial leaflet 101 is transferred from the delivery state 102 to the activated state 103. Alternative, or in addition to, the artificial leaflet 101 may also comprise shape memory material so in the first temperature the artificial leaflet 101 is in the rolled form (delivery state 102) and in the second temperature the artificial leaflet 101 is rolled open to the activated state 103. In addition, according to an embodiment the artificial leaflet 101 can be rolled around the pusher 105 and opened into the activated state by turning the pusher 105 around a longitudinal axis of the pusher 105.

In addition, it is to be noted that the artificial leaflet 101 illustrated in FIGS. 23-25 may comprise the slidable fixing means 106, whereupon even the rolled artificial leaflet 101 can in addition to be pressed much smaller packet during delivery to transfer the distal and proximal end 101A, 101B of the artificial leaflet 101 closer to each other and e.g. just before rolling the artificial leaflet 101 transfer the distal and proximal ends 101A, 101B farther from each other. Further, it is to be noted that according to an embodiment the slidable fixing means 106 are optional and the artificial leaflet 101 is just rolled in in the delivery state 102 and the roll is opened to transfer the artificial leaflet 101 to the activated state so that the distance between the distal and proximal ends of the artificial leaflet is essentially constant.

FIG. 26 illustrates an example of the tissue anchoring device in an elongated form of the delivery state. In addition, FIG. 26 illustrates an example where the arrangement has two artificial leaflets 101. In the example they are in the activated states, but it is be understood that they can be delivered to the heart in the delivery states 102 and opened or transferred to the activated states 103 similarly as is discussed in connection with another embodiment in this document.

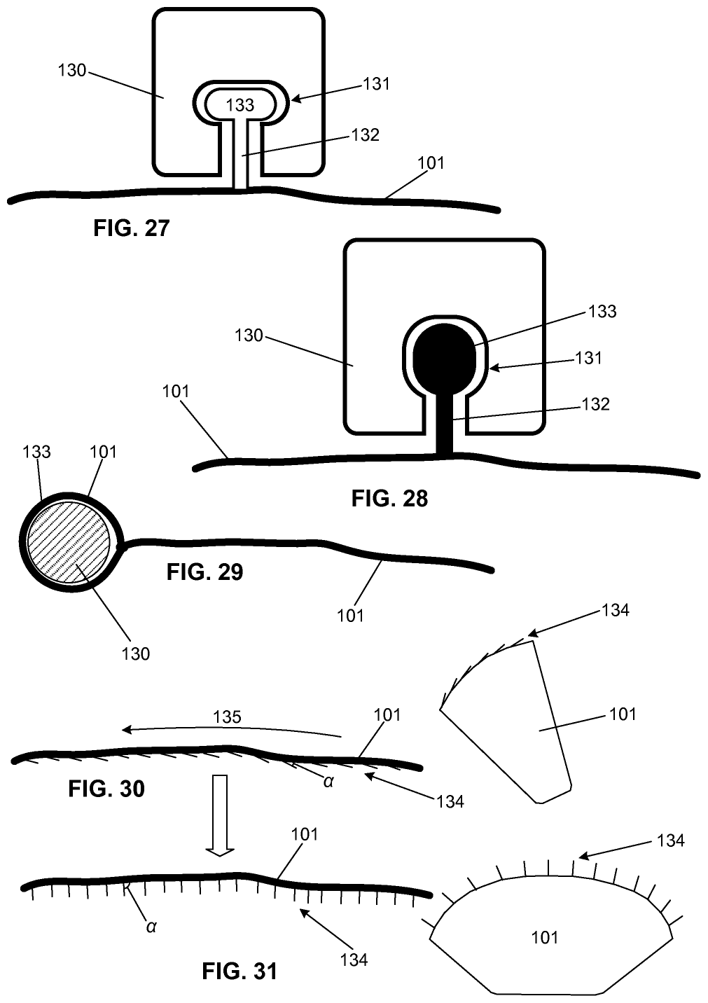

FIGS. 27-28 illustrates examples of the slidable fixing means 106 according to an advantageous embodiment of the invention. In this embodiment the tissue anchoring device 130 comprises a groove 131 and the artificial leaflet comprises protrusion members 132, where each of the protrusion member comprises an enlarged end portion 133, as can be seen in FIGS. 27-28. The enlarged end portions 133 advantageously slide along the groove 131 of the tissue anchoring device when the artificial leaflet is expanded to the activate state, for example. In FIGS. 28-28 the enlarged end portion 133 comprises a hanger which slides in the groove and at the same fix the artificial leaflet to the tissue anchoring device.

FIG. 29 illustrates an alternatively embodiment, where the edge portion of the artificial leaflet 101 is folded so to have a thick portion and thus having enlarged end portion 133 which can be introduced to the groove 131 and to slide in the groove and at the same fix the artificial leaflet 101 to the tissue anchoring device 130.

FIGS. 30-31 illustrates examples of the securing members 134 of the artificial leaflet 101 for securing the artificial leaflet 101 according to an advantageous embodiment of the invention to the tissue. The securing members 134 can be implemented by sharp tooths or spikes, for example. In FIG. 30 the artificial leaflet 101 is in the delivery state, where the angle a is small so that the free ends of the securing members point more towards the proximal end of the tissue anchoring device, and thus the artificial leaflet 101 can be moved easily in the direction 135. Then, when the artificial leaflet 101 is expanded to the activated state, the angles a of the securing members 134 are advantageously increased so that the free ends of the securing members point more towards the tissue thereby biting down to the tissue and securing the artificial leaflet 101 to the tissue, as is depicted in FIG. 31. The change of the angle of the securing members may be achieved by the shape memory material, for example.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

For example, it is to be noted that the distal and proximal ends 101A, 101B of the artificial leaflet 101 can also be fixed another portion of the tissue anchoring device 130 during delivery than only to the distal or proximal portions 130A, 130B, such as e.g. to a mid area between the distal or proximal portions 130A, 130B, whereupon the distal end 101A of the artificial leaflet 101 is delivered further towards the distal area 22A of the biological leaflet and the proximal end 101B of the of the artificial leaflet 101 is moved backwards towards the proximal area 22B of the biological leaflet and so to transfer the artificial leaflet 101 from the delivery state to the activated state. The transfer can be done correspondingly as discussed in connection with another embodiments in this document, and for example if the artificial leaflet 101 is in the tensioned position in the delivery state or comprises shape memory material, it will unfold and open from the mid area of the tissue anchoring device both towards the distal and proximal areas 22A, 22B of the biological leaflet.

The features recited in dependent claims are mutually freely combinable unless otherwise explicitly stated. In particularly, even if only one artificial leaflet is described above, it is also clear that two artificial leaflets can be delivered in manner described for one artificial leaflet.

1. A medical arrangement for improving function of a heart valve, the heart valve comprising valve tissue, including an annulus at an outer section of said valve tissue and in a normal healthy state a plurality of biological leaflets at an inner section of said valve tissue,
wherein the medical arrangement comprises:
a tissue anchoring device to be introduced around or to the annulus so that the tissue anchoring device surrounds at least a circular portion of the annulus,
an artificial leaflet to be introduced to the inner section of said valve tissue, wherein
said artificial leaflet comprises a delivery state and an activated state, wherein
in said delivery state the leaflet has a reduced form so that an area of the leaflet is smaller than in said activated state and wherein said artificial leaflet is introduced with the tissue anchoring device, and
in the activated state the leaflet has an expanded form so that the area of the leaflet is bigger than in said delivery state and wherein the leaflet in the expanded form covers at least a portion of the biological leaflets,
wherein the artificial leaflet is transferable from said delivery state to said activated state when the tissue anchoring device is introduced to surround at least a circular portion of the annulus.

2. A medical arrangement of claim 1, wherein the artificial leaflet has distal and proximal ends and wherein the artificial leaflet is in a folded form in said delivery state so that a distance between the distal and proximal ends of the artificial leaflet is smaller than in said activated state.

3. A medical arrangement of claim 2, wherein the artificial leaflet is fixed to the tissue anchoring device or a pusher so that the tissue anchoring device or the pusher is configured to transport the artificial leaflet during introduction of said tissue anchoring device or the pusher and wherein
the proximal end of the artificial leaflet is configured to be released from the tissue anchoring device or the pusher when the proximal end of the artificial leaflet is at a proximal area of the biological leaflet, and so that the distal end of the artificial leaflet will drag the rest of the artificial leaflet with the tissue anchoring device or the pusher and thereby transfer the artificial leaflet from said delivery state to said activated state at the same when the tissue anchoring device is introduced to surround at least a circular portion of the annulus or the pusher is elongated over the portion of the annulus.

4. A medical arrangement of claim 2, wherein the artificial leaflet is fixed to the tissue anchoring device or a pusher so that the tissue anchoring device or the pusher is configured to transport the distal end of the artificial leaflet via a proximal area to a distal area of the biological leaflet during introduction of the tissue anchoring device or the pusher, whereupon the proximal end of the artificial leaflet is configured to be released from the tissue anchoring device or the pusher when the distal end of the artificial leaflet is at the distal area of the biological leaflet and the proximal end of the artificial leaflet is configured to be retracted back towards the proximal area of the biological leaflet.

5. A medical arrangement of claim 4, wherein the proximal end of the artificial leaflet is configured to be coupled in a detachable manner to a retraction device, such as a yarn or a catheter or the like, operable outside the heart so that when the retraction device is operated outside the heart the proximal end of the artificial leaflet is configured to be retracted back towards the proximal area of the biological leaflet and detached from retraction device the when the proximal end of the artificial leaflet is essentially at a proximal area of the biological leaflet and thereby transfer the artificial leaflet from said delivery state to said activated state.

6. A medical arrangement of claim 2, wherein a pusher is used to introduce the tissue anchoring device together with the artificial leaflet and wherein the proximal end of the artificial leaflet is configured to be released when the proximal end of the artificial leaflet is at a proximal area of the biological leaflet, and so that the distal end of the artificial leaflet will drag the rest of the artificial leaflet with the tissue anchoring device and the pusher and thereby transfer the artificial leaflet from said delivery state to said activated state at the same when the tissue anchoring device is introduced to surround at least a circular portion of the annulus by the pusher.

7. A medical arrangement of claim 4, wherein the pusher is used for introducing the tissue anchoring device and the proximal end of the artificial leaflet is retraced back towards the proximal area of the biological leaflet by the pusher and released from the pusher when the proximal end of the artificial leaflet is essentially at a proximal area of the biological leaflet and thereby drag the artificial leaflet with the pusher and transfer the artificial leaflet from said delivery state to said activated state by the same back and forth movement when the tissue anchoring device is introduced to surround at least a circular portion of the annulus.

8. A medical arrangement of claim 1, wherein the artificial leaflet comprises a stretch material so that in said delivery state the artificial leaflet is in a rest position and in said activated state the artificial leaflet is stretched to said expanded form or wherein in the said delivery state the artificial leaflet is in a tensioned position having potential energy and in said activated state the potential energy of the tensioned artificial leaflet is released to cause the artificial leaflet to transform to the expanded form.

9. A medical arrangement of claim 1, wherein the artificial leaflet comprises a shape memory material having a first shape, such as the delivery state in a first temperature, and the second shape, such as the activated state in a second temperature, whereupon the artificial leaflet is configured to take said activated state when it is exposed to said second temperature and one of the distal or proximal end is released.

10. A medical arrangement of claim 1, wherein the artificial leaflet is configured to be rolled next to the tissue anchoring device or a pusher in said delivery state and wherein the artificial leaflet is configured to be rolled open from the tissue anchoring device or the pusher when the tissue anchoring device is introduced around the annulus.

11. A medical arrangement of claim 10, wherein the artificial leaflet comprises shape memory material having a first shape, such as the delivery state in a first temperature, and the second shape, such as the activated state in a second temperature, whereupon the artificial leaflet is configured to roll open from the tissue anchoring device or the pusher and take said activated state when it is exposed to said second temperature and a free end of the artificial leaflet to be pointed towards a free end of the biological leaflet is released from the tissue anchoring device or the pusher.

12. A medical arrangement of claim 1, wherein the artificial leaflet is configured to be rolled around a pusher in said delivery state and wherein the artificial leaflet is configured to be rolled open around the pusher when the pusher is rotated around its longitudinal axis.

13. A medical arrangement of claim 2, wherein the tissue anchoring device comprises a securing member, such as a sharp tooth, so to secure the artificial leaflet to said activated state when the artificial leaflet is expanded to said activated state.

14. A medical arrangement of claim 2, wherein the artificial leaflet comprises a slidable fixing means, such as a loop or the like, which is configured to slide along the tissue anchoring device when the artificial leaflet is transformed to the activated state from the delivery state.

15. A medical arrangement of claim 2, wherein the tissue anchoring device comprises a groove, and the slidable fixing means of the artificial leaflet comprises protrusion members, which are configured to slide in the groove of the tissue anchoring device and at the same to secure the artificial leaflet to the tissue anchoring device when the artificial leaflet is transformed to the activated state from the delivery state.

16. A medical arrangement of claim 2, wherein the tissue anchoring device is configured to press the artificial leaflet between the annulus and the tissue anchoring device by a sprig force of the tissue anchoring device and thereby secure and prevent the movement of the artificial leaflet between the delivery and activated states after the artificial leaflet is expanded to said activated state.

17. A medical arrangement of claim 1, wherein the artificial leaflet comprises securing members, such as sharp teeth or spikes, to secure the artificial leaflet to the tissue when the artificial leaflet is expanded to the activated state.

18. A medical arrangement of claim 1, wherein a pusher is used to introduce the artificial leaflet
wherein the distal end of the artificial leaflet is coupled with the distal end of the pusher, and wherein the distal end of the artificial leaflet is configured to be released from the pusher when the distal end of the artificial leaflet is introduced to the distal area of the biological leaflet,
and wherein
the proximal end of the artificial leaflet is configured to be released or left to the proximal area of the biological leaflet from the pusher and when the proximal end of the artificial leaflet is at the proximal area of the biological leaflet and thereby transfer the artificial leaflet from said delivery state to said activated state at the same when the distal end of the artificial leaflet is delivered towards the distal area of the biological leaflet, or
the proximal end of the artificial leaflet is configured to be retracted back towards the proximal area of the biological leaflet by the pusher after the distal end of the artificial leaflet is introduced by the pusher to the distal area of the biological leaflet and thereby transfer the artificial leaflet from said delivery state to said activated state at the same when the proximal end of the artificial leaflet is retracted back towards the proximal area of the biological leaflet.

19. A medical arrangement of claim 1, wherein
the proximal end of the artificial leaflet is configured to be retracted back towards the proximal area of the biological leaflet by a catheter after the distal end of the artificial leaflet is introduced to the distal area of the biological leaflet and thereby transfer the artificial leaflet from said delivery state to said activated state at the same when the proximal end of the artificial leaflet is retracted back towards the proximal area of the biological leaflet by the catheter.

20. A method for improving function of a heart valve, the heart valve comprising valve tissue, including an annulus at an outer section of said valve tissue and in a normal healthy state a plurality of biological leaflets at an inner section of said valve tissue,
wherein the method comprises:
introducing a tissue anchoring device around or to the annulus so that the tissue anchoring device surrounds at least a circular portion of the annulus,
introducing an artificial leaflet to the inner section of said valve tissue, wherein
said artificial leaflet comprises a delivery state and an activated state, wherein
in said delivery state the leaflet has a reduced form so that an area of the leaflet is smaller than in said activated state and wherein said artificial leaflet is introduced with the tissue anchoring device, and
in the activated state the leaflet has an expanded form so that the area of the leaflet is bigger than in said delivery state and wherein the leaflet in the expanded form covers at least a portion of the biological leaflets,
wherein the artificial leaflet is transferred from said delivery state to said activated state when the tissue anchoring device is introduced to surround at least a circular portion of the annulus.
